(19) United States
(12) Patent Application Publication
(10) Pub. No.: US 2025/0243147 A1
(43) Pub. Date: Jul. 31, 2025

(54) ALICYCLIC ALIPHATIC 1,3-DICARBONYL DERIVATIVES USEFUL AS FRAGRANCES

(71) Applicant: GIVAUDAN SA, VERNIER (CH)

(72) Inventor: Nicolas COCITO ARMANINO, Kemptthal (CH)

(21) Appl. No.: 18/705,043

(22) PCT Filed: Oct. 20, 2022
(86) PCT No.: PCT/EP2022/079187

§ 371 Date: Apr. 26, 2024

(30) Foreign Application Priority Data
Oct. 27, 2021 (GB) 2115444.8

(51) Int. Cl. C07C 69/716; A61K 8/37; A61Q 13/00

(52) U.S. Cl. CPC C07C 69/716; A61K 8/37; A61Q 13/00

(57) ABSTRACT

Disclosed are alicyclic aliphatic 1,3-dicarbonyl derivatives of formula (I) possessing musk odor characteristics.

The present invention is concerned with fragrance ingredients and with fragrance preparations, for imparting desired odor notes to consumer products, in particular it is concerned with a novel class of acyclic aliphatic 1,3-dicarbonyl derivatives of formula (I) possessing musk odor characteristics.

In the fragrance and flavor industry, perfumers and flavorists are continually looking for new compounds possessing unique odor characteristics.

Acyclic aliphatic 1,3 dicarbonly derivatives are known in the art. As one example one may cite 3-methyl-2,4-nonanedione A (Tee Dione™; CAS 113486-29-6) possessing a straw-like and fruity flavor (J. Agric. Food Chem. 2001, 49, 3864-3866). As another example one may cited 3,5-decanedione B possessing marine-like, modern green-like fragrance characteristics (US 2003/0092599).

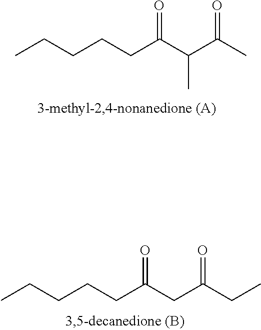

Another acyclic aliphatic 1,3-dicarbonly derivative is geranyl acetoacetate C (geranyl beta-ketobutyrate; CAS 10032-00-5) possessing a sweet fruity, winey-green, fermented Apple-like odor (Arctander, 1969, No 1431).

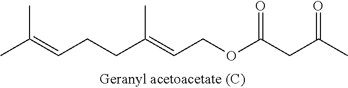 [SMILES: CC(=O)CC(=O)OC/C=C(\C)CCC=C(C)C]

Surprisingly, we have now found that certain acyclic aliphatic 1,3-dicarbonyl derivatives possess musk odor characteristics. This new class of compounds has not been described in the literature.

Thus there is provided in accordance with a first aspect of the present invention the use as fragrance of a compound of formula (I) (or a mixture thereof)

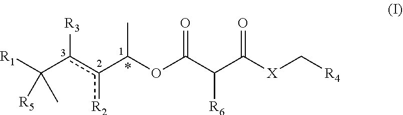

wherein
R₁ and R₃ are independently selected from hydrogen, methyl and ethyl;
R₂ is hydrogen, methyl, ethyl, or methylene;
R₄ is H, or C₁-C₄ alkyl;
R₅ is H, or methyl;
R₆ is selected from hydrogen and methyl;
X is selected from the group consisting of the bivalent residues >CH2, >O, >CHMe, >CMe₂; and
the bond between C-2 and R₂ is a single bond, and the bond between C-2 and C-3 together with the dotted line represents a double bond; or
the bond between C-2 and C-3 is a single bond, and the bond between C-2 and R₂ together with the dotted line represents a double bond; or
the bond between C-2 and R₂ is a single bond, and the bond between C-2 and C-3 is a single bond.

Non-limiting examples are compounds of formula (Ia)

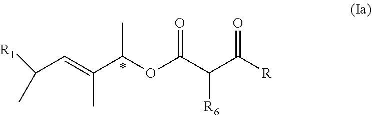

wherein
R₁ is selected from hydrogen, methyl and ethyl;
R₆ is selected from hydrogen, methyl and ethyl; and
R is a hydrocarbon residue comprising 1-6 carbon atoms (e.g. 2-4 carbon atoms), optionally comprising one oxygen atom.

Further non-limiting examples are compounds of formula (I) (which encompasses compounds of formula (Ia) wherein R₁ and R₂ are methyl and R₃ and R₅ are hydrogen.

Further non-limiting examples are compounds of formula (Ia) wherein R is selected from C₂-C₆ alkyl (e.g. propyl, butyl), and C₂-C₅ alkoxy

Further non-limiting examples are compounds of formula (Ia) wherein R₁ is hydrogen or methyl and R₆ is hydrogen or methyl.

Further non-limiting examples are compounds of formula (Ia) wherein R is selected from propyl, butyl, and ethoxy.

Further non-limiting examples are compounds of formula (Ia) wherein R is propyl.

Further non-limiting examples are compounds of formula (Ia) wherein the double bond is in E-configuration.

Preferred compounds of formula (I) are selected from the group consisting of 3,5-dimethylhex-3-en-2-yl 3-oxohexanoate (e.g. (S,E)-3,5-dimethylhex-3-en-2-yl 3-oxohexanoate); 3,5-dimethylhex-3-en-2-yl 2-methyl-3-oxohexanoate; (E)-3-methylhex-3-en-2-yl 3-oxohexanoate; (E)-3,5-dimethylhex-3-en-2-yl 4-methyl-3-oxohexanoate; and 3,5-dimethylhex-3-en-2-yl ethyl malonate.

The compounds according to the present invention comprise one or more chiral centres and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methodology known in the art, e.g. preparative HPLC and GC or by stereoselective syntheses.

In one particular embodiment the compounds of formula (I) (which encompasses compounds of formula (Ia)) are racemic mixtures.

In one particular embodiment the compounds of formula (I) (which encompasses compounds of formula (Ia)) are compounds wherein the position designated with a * have the absolute configuration of S.

The compounds of formula (I) (which encompasses compounds of formula (Ia)) may be used alone, as isomeric mixture thereof, or in combination with known odorant molecules selected from the extensive range of natural products, and synthetic molecules currently available, such as essential oils, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, and other auxiliary agents commonly used in the art.

As used herein, “carrier material” means a material which is practically neutral from a odorant point of view, i.e. a material that does not significantly alter the organoleptic properties of odorants.

The term “auxiliary agent” refers to ingredients that might be employed in a fragrance composition for reasons not specifically related to the olfactive performance of said composition. For example, an auxiliary agent may be an ingredient that acts as an aid to processing a fragrance ingredient or ingredients, or a composition containing said ingredient(s), or it may improve handling or storage of a fragrance ingredient or composition containing same. It might also be an ingredient that provides additional benefits such as imparting color or texture. It might also be an ingredient that imparts light resistance or chemical stability to one or more ingredients contained in a fragrance composition. A detailed description of the nature and type of adjuvants commonly used in fragrance compositions containing same cannot be exhaustive, but it has to be mentioned that said ingredients are well known to a person skilled in the art.

As used herein, ‘fragrance composition’ means any composition comprising a compound of formula (I) (which encompasses compounds of formula (Ia)), or a mixture thereof and a base material, e.g. a diluent conventionally used in conjunction with odorants, such as diethyl phthalate (DEP), dipropylene glycol (DPG), isopropyl myristate (IPM), pentane-1,2-diol, triethyl citrate (TEC) and alcohol (e.g. ethanol). Optionally, the composition may comprise an anti-oxidant adjuvant. Said anti-oxidant may be selected from Tinogard® TT (BASF), Tinogard® Q (BASF), Tocopherol (including its isomers, CAS 59-02-9; 364-49-8; 18920-62-2; 121854-78-2), 2,6-bis(1,1-dimethylethyl)-4-methylphenol (BHT, CAS 128-37-0) and related phenols, hydroquinones (CAS 121-31-9).

The following list comprises examples of known odorant molecules, which may be combined with a compound of formula (I) (which encompasses compounds of formula (Ia)), or a mixture thereof:
essential oils and extracts, e.g. castoreum, costus root oil, oak moss absolute, geranium oil, tree moss absolute, basil oil, fruit oils, such as bergamot oil and mandarine oil, myrtle oil, palmarose oil, patchouli oil, petitgrain oil, jasmine oil, rose oil, sandalwood oil, wormwood oil, lavender oil and/or ylang-ylang oil;
alcohols, e.g. cinnamic alcohol ((E)-3-phenylprop-2-en-1-ol); cis-3-hexenol ((Z)-hex-3-en-1-ol); citronellol (3,7-dimethyloct-6-en-1-ol); dihydro myrcenol (2,6-dimethyloct-7-en-2-ol); Ebanol™ ((E)-3-methyl-5-(2,2,3-trimethylcyclopent-3-en-1-yl)pent-4-en-2-ol); eugenol (4-allyl-2-methoxyphenol); ethyl linalool ((E)-3,7-dimethylnona-1,6-dien-3-ol); farnesol ((2E,6Z)-3,7,11-trimethyldodeca-2,6,10-trien-1-ol); geraniol ((E)-3,7-dimethylocta-2,6-dien-1-ol); Super Muguet™ ((E)-6-ethyl-3-methyloct-6-en-1-ol); linalool (3,7-dimethylocta-1,6-dien-3-ol); menthol (2-isopropyl-5-methylcyclohexanol); Nerol (3,7-dimethyl-2,6-octadien-1-ol); phenyl ethyl alcohol (2-phenylethanol); Rhodinol™ (3,7-dimethyloct-6-en-1-ol); Sandalore™ (3-methyl-5-(2,2,3-trimethylcyclopent-3-en-1-yl)pentan-2-ol); terpineol (2-(4-methylcyclohex-3-en-1-yl)propan-2-ol); or Timberol™ (1-(2,2,6-trimethylcyclohexyl)hexan-3-ol); 2,4,7-trimethylocta-2,6-dien-1-ol, and/or [1-methyl-2(5-methylhex-4-en-2-yl)cyclopropyl]-methanol;
aldehydes and ketones, e.g. anisaldehyde (4-methoxybenzaldehyde); alpha amyl cinnamic aldehyde (2-benzylideneheptanal); Georgywood™ (1-(1,2,8,8-tetramethyl-1,2,3,4,5,6,7,8-octahydronaphthalen-2-yl)ethanone); Hydroxycitronellal (7-hydroxy-3,7-dimethyloctanal); Iso E Super® (1-(2,3,8,8-tetramethyl-1,2,3,4,5,6,7,8-octahydronaphthalen-2-yl)ethanone); Isoraldeine® ((E)-3-methyl-4-(2,6,6-trimethylcyclohex-2-en-1-yl)but-3-en-2-one); Hedione™ (methyl 3-oxo-2-pentylcyclopentaneacetate); 3-(4-isobutyl-2-methylphenyl)propanal; maltol; methyl cedryl ketone; methylionone; verbenone; and/or vanillin;
ether and acetals, e.g. Ambrox® (3a,6,6,9a-tetramethyl-2,4,5,5a,7,8,9,9b-octahydro-1H-benzo[e][1]benzofuran); geranyl methyl ether ((2E)-1-methoxy-3,7-dimethylocta-2,6-diene); and/or Spirambrene® (2′,2′,3,7,7-pentamethylspiro[bicyclo[4.1.0]heptane-2,5′-[1,3]dioxane]);
esters and lactones, e.g. benzyl acetate; cedryl acetate ((1S,6R,8aR)-1,4,4,6-tetramethyloctahydro-1H-5,8a-methanoazulen-6-yl acetate); delta-decalactone (6-pentyltetrahydro-2H-pyran-2-one); Helvetolide® (2-(1-(3,3-dimethylcyclohexyl)ethoxy)-2-methylpropyl propionate); delta-undecalactone (5-heptyloxolan-2-one); and/or vetiveryl acetate ((4,8-dimethyl-2-propan-2-ylidene-3,3a,4,5,6,8a-hexahydro-1H-azulen-6-yl) acetate);
macrocycles, e.g. Ambrettolide ((Z)-oxacycloheptadec-10-en-2-one); ethylene brassylate (1,4-dioxacycloheptadecane-5,17-dione); and/or Exaltolide® (16-oxacyclohexadecan-1-one); and
heterocycles, e.g. isobutylquinoline (2-isobutylquinoline).

Thus there is provided in a further aspect of the invention a fragrance composition comprising a compound of formula (I) (which encompasses compounds of formula (Ia)).

The compounds of formula (I) (which encompasses compounds of formula (Ia)) may be used in a broad range of fragranced articles, e.g. in any field of fine and functional perfumery, such as perfumes, air care products, household products, laundry products, body care products and cosmetics. The compound can be employed in widely varying amounts, depending upon the specific article and on the nature and quantity of other odorant ingredients. The proportion is typically from 0.00001 to 3 weight percent of the article. In one embodiment, the compound may be employed in a fabric softener in an amount from 0.0001 to 0.3 weight percent (e.g. 0.001 to 0.1 including 0.05 weight %). In another embodiment, the compound may be used in fine perfumery in amounts from 0.01 to 30 weight percent (e.g. up to about 10 or up to 20 weight percent), more preferably between 0.01 and 5 weight percent (e.g. 0.01 to 0.1 weight percent). However, these values are given only by way of example, since the experienced perfumer may also achieve effects or may create novel accords with lower or higher concentrations.

The compounds of formula (I) (which encompasses compounds of formula (Ia)) may be employed in a consumer product base simply by directly mixing the compound, or a fragrance composition comprising a compound of formula (I) (which encompasses compounds of formula (Ia)), or a mixture thereof, with the consumer product base, or it may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, and then mixed with the consumer product base.

Thus, the invention additionally provides a method of manufacturing a fragranced article, comprising the incorporation a compound of formula (I) (which encompasses compounds of formula (Ia)), or a mixture thereof as a fragrance ingredient, either by directly admixing to the consumer product base or by admixing a fragrance composition comprising a compound of formula (I) (which encompasses compounds of formula (Ia)), or a mixture thereof, which may then be mixed with a consumer product base, using conventional techniques and methods. Through the addition of an olfactory acceptable amount of a compound of formula (I) (which encompasses compounds of formula (Ia)), or a mixture thereof the odor notes of a consumer product base will be improved, enhanced, or modified.

Thus, the invention furthermore provides a method for improving, enhancing or modifying a consumer product base by means of the addition thereto of an olfactory acceptable amount of a compound of formula (I) (which encompasses compounds of formula (Ia)), or a mixture thereof.

There is provided in a further aspect of the present invention a fragranced article comprising:
a) a compound of formula (I)

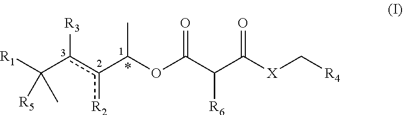

wherein
R₁ and R₃ are independently selected from hydrogen, methyl and ethyl;
R₂ is hydrogen, methyl, ethyl, or methylene;
R₄ is H, or C₁-C₄ alkyl;
R₅ is H, or methyl;
R₆ is selected from hydrogen and methyl;
X is selected from the group consisting of the bivalent residues >CH2, >O, >CHMe, >CMe₂; and
the bond between C-2 and R₂ is a single bond, and the bond between C-2 and C-3 together with the dotted line represents a double bond; or
the bond between C-2 and C-3 is a single bond, and the bond between C-2 and R₂ together with the dotted line represents a double bond; or
the bond between C-2 and R₂ is a single bond, and the bond between C-2 and C-3 is a single bond;
and
b) a consumer product base.

In one particular embodiment the fragranced article comprises a compound of formula (Ia)

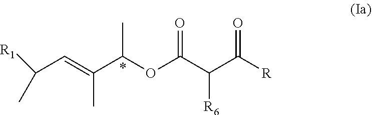

wherein
R₁ is selected from hydrogen, methyl and ethyl;
R₆ is selected from hydrogen, methyl and ethyl; and
R is a hydrocarbon residue comprising 1-6 carbon atoms (e.g. 2-4 carbon atoms), optionally comprising one oxygen atom.

As used herein, ‘consumer product base’ means a composition for use as a consumer product to fulfill specific actions, such as cleaning, softening, and caring or the like. Examples of such products include fine perfumery, e.g. perfume and eau de toilette; fabric care, household products and personal care products such as cosmetics, laundry care detergents, rinse conditioner, personal cleansing composition, detergent for dishwasher, surface cleaner; laundry products, e.g. softener, bleach, detergent; body-care products, e.g. shampoo, shower gel; air care products (includes products that contain preferably volatile and usually pleasant-smelling compounds which advantageously can even in very small amounts mask unpleasant odors). Air fresheners for living areas contain, in particular, natural and synthetic essential oils such as pine needle oils, citrus oil, eucalyptus oil, lavender oil, and the like, in amounts for example of up to 50% by weight. As aerosols they tend to contain smaller amounts of such essential oils, by way of example less than 5% or less than 2% by weight, but additionally include compounds such as acetaldehyde (in particular, <0.5% by weight), isopropyl alcohol (in particular, <5% by weight), mineral oil (in particular, <5% by weight), and propellants.

Cosmetic products include:
(a) cosmetic skincare products, especially bath products, skin washing and cleansing products, skincare products, eye makeup, lip care products, nail care products, intimate care products, foot care products;
(b) cosmetic products with specific effects, especially sunscreens, tanning products, de-pigmenting products, deodorants, antiperspirants, hair removers, and shaving products;
(c) cosmetic dental-care products, especially dental and oral care products, tooth care products, cleaners for dental prostheses, adhesives for dental prostheses; and
(d) cosmetic hair care products, especially hair shampoos, hair care products, hair setting products, hair-shaping products, and hair coloring products.

This list of products is given by way of illustration, and is not to be regarded as being in any way limiting.

In one particular embodiment the consumer product base is selected form fine perfumery, and personal care products, including deodorants, hair care products, soaps, and the like.

In a further particular embodiment the consumer product base is selected from fabric care products, including fabric softener, and home care products, including air fresheners, dish washers and the like.

The compounds of formula (I) (which encompasses compounds of formula (Ia)) have not been described in literature, and thus are novel in its own.

Thus there is provided in accordance with a further aspect of the present invention compounds of formula (I)

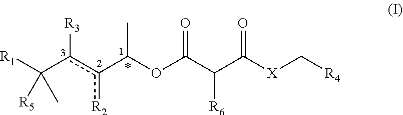

wherein
R₁ and R₃ are independently selected from hydrogen, methyl and ethyl;
R₂ is hydrogen, methyl, ethyl, or methylene;
R₄ is H, or C₁-C₄ alkyl;
R₅ is H, or methyl;
R₆ is selected from hydrogen and methyl;
X is selected from the group consisting of the bivalent residues >CH2, >O, >CHMe, >CMe₂; and
the bond between C-2 and R₂ is a single bond, and the bond between C-2 and C-3 together with the dotted line represents a double bond; or
the bond between C-2 and C-3 is a single bond, and the bond between C-2 and R₂ together with the dotted line represents a double bond; or
the bond between C-2 and R₂ is a single bond, and the bond between C-2 and C-3 is a single bond.

The compounds of formula (I) may, for example, be synthesized by formation of the ester bond. This can be done by reacting the corresponding alcohol with an appropriate ethyl 3-oxoalkanoate or malonate under conditions of trans-esterification. The reaction is conducted in a way that the released alcohol (e.g. ethanol) can be distilled off from the reaction mixture, driving the equilibrium of the trans-esterification towards the desired product. Accordingly, the reaction should be carried out in an appropriately high-boiling solvent such as but not limited to Toluene or without solvent, and at an appropriate temperature, such as between 70° C. and 180° C. The reaction may benefit from a catalyst that promotes exchange of the ester alcohols such as but not limited to cesium fluoride. Substitution at the 2-position of the resulting ketoesters or malonates (i.e. between both carbonyl carbons) can be effected by alkylation. For that, the ketoester or malonate is treated with a base such as but not limited to sodium hydride, in a suitable solvent such as tetrahydrofuran (THF), followed by quenching with the appropriate alkyl-halide, such as methyl iodide. Alternative methods of preparation are also possible and will be clear to someone skilled in the art of organic synthesis.

The invention is now further described with reference to the following non-limiting examples. These examples are for the purpose of illustration only and it is understood that variations and modifications can be made by one skilled in the art.

### EXAMPLE 1: 3,5-DIMETHYLHEX-3-EN-2-YL 3-OXOHEXANOATE

3,5-Dimethylhex-3-en-2-ol (12.7 g, 99.05 mmol), ethyl 3-oxohexanoate (15.66 g, 99.05 mmol) and cesium fluoride (1.32 g, 9.9 mmol) were mixed in anhydrous Toluene (130 mL). The resulting reaction mixture was stirred at 110° C. for 52 h under nitrogen atmosphere. The reaction mixture was then diluted with water (100 mL) and extracted in Ethyl acetate (EtOAc) (2×300 mL), washed with brine, dried over anhydrous sodium sulphate (Na₂SO₄) and concentrated under reduced pressure to obtain the crude. The crude product was purified by column chromatography on silica gel eluting with a gradient of ethyl acetate in heptane to give 3,5-dimethylhex-3-en-2-yl 3-oxohexanoate (8.8 g, 37% yield) as a colorless liquid.

¹H NMR (400 MHz, CDCl3, 298 K, mixture of E and Z isomers) δ (ppm)=5.35-4.92 (m, 2H), 3.45-3.35 (m, 2H), 2.76-2.42 (m, 2H), 1.71-1.54 (m, 5H), 1.37-1.27 (m, 3H), 1.00-0.88 (m, 9H).

¹³C NMR (101 MHz, CDCl3, 298 K, mixture of E and Z isomers) b (ppm)=202.8, 166.5, 136.6, 135.5, 131.3, 76.7, 70.2, 49.7, 49.5, 44.8, 44.8, 26.8, 26.7, 23.1, 22.7, 19.0, 18.9, 17.5, 16.9, 13.5, 11.8.

MS (El, 70 eV): 240 (1, [M]⁺*), 197 (2), 128 (16), 113 (91), 110 (76), 95 (88), 71 (100), 43 (52).

Odour description (10% solution in DPG on paper blotter, 4 h): musky, powdery, fatty, slightly green, pear skin.

### EXAMPLE 2: (S,E)-3,5-DIMETHYLHEX-3-EN-2-YL 3-OXOHEXANOATE

Was prepared in the same way as Example 1, using (S,E)-3,5-dimethylhex-3-en-2-ol (3.00 g, 23.4 mmol), ethyl 3-oxohexanoate (3.70 g, 23.4 mmol) and CsF (355 mg, 2.34 mmol) in Toluene (25 mL) to give (S,E)-3,5-dimethylhex-3-en-2-yl 3-oxohexanoate (1.70 g, 30% yield) as a colorless liquid.

¹H NMR (400 MHz, CDCl3, 298 K) b (ppm)=5.33-5.24 (m, 2H), 3.40 (s, 2H), 2.51 (t, J=7.2 Hz, 2H), 1.67-1.59 (m, 5H), 1.31 (d, J=6.5 Hz, 3H), 0.97-0.89 (m, 9H).

¹³C NMR (101 MHz, CDCl3, 298 K) b (ppm)=202.9, 166.5, 135.6, 131.3, 76.8, 49.7, 44.8, 26.8, 22.7, 18.9, 16.9, 13.6, 11.8.

Optical rotation: [α]_{D}²⁴=−29.497 deg*mL*g⁻¹*dm⁻¹ (c 1.83, EtOH); [α]₄₀₅²⁴=−74.915 deg*mL*g⁻¹*dm⁻¹ (c 1.83, EtOH).

Odour description (10% solution in DPG on paper blotter, 4 h): musky, powdery, radiant, fatty, green, fruity, pear skin.

### EXAMPLE 3: 3,5-DIMETHYLHEX-3-EN-2-YL 2-METHYL-3-OXOHEXANOATE

NaH (0.16 g, 60 wt-% in mineral oil, 3.9 mmol) was mixed with THF (tetrahydrofuran) (3 mL). A solution of 3,5-dimethylhex-3-en-2-yl 3-oxohexanoate (0.85 g, 3.5 mmol) in THF (5 mL) was added dropwise and the resulting reaction mixture was stirred for 20 min at ambient temperature. A solution of iodomethane (0.55 g, 0.24 mL, 3.9 mmol) in THF (2 mL) was added. The reaction mixture was stirred at ambient temperature for 90 min then poured into iced 1M HCl solution (100 mL) and extracted with MTBE (methyl tert-butyl ether) (2×100 mL), washed with water (2×100 mL) and brine (100 mL), dried over MgSO₄, filtered and concentrated. The crude product was purified by column chromatography on silica gel eluting with a gradient of MTBE in heptane to give 3,5-dimethylhex-3-en-2-yl 2-methyl-3-oxohexanoate (0.9 g, 97% purity, 57% yield) as a colorless liquid.

¹H NMR (400 MHz, CDCl3, 298 K, mixture of stereoisomers) b (ppm)=5.89-5.47 (m, 0.5H), 5.35-5.22 (m, 1H), 5.13-4.81 (m, 1H), 3.57-3.40 (m, 1H), 2.74-2.40 (m, 3H), 1.71-1.56 (m, 6H), 1.37-1.28 (m, 5H), 1.01-0.87 (m, 9H). ¹³C NMR (101 MHz, CDCl3, 298 K mixture of stereoisomers) b (ppm)=205.7, 169.8, 135.7, 135.5, 77.2, 76.6, 76.6, 53.2, 53.1, 43.2, 43.1, 26.7, 26.7, 23.4, 22.7, 18.9, 18.8, 17.4, 17.0, 17.0, 13.6, 13.6, 12.7, 11.8.

MS (El, 70 eV): 155 (3), 145 (5), 127 (77), 110 (57), 95 (53), 71 (100), 43 (51).

Odour description (10% solution in DPG on paper blotter, 4 h): musky, animalic, warm.

### EXAMPLE 4: (E)-3-METHYLHEX-3-EN-2-YL 3-OXOHEXANOATE

A mixture of (E)-3-methylhex-3-en-2-ol (3.40 g, 29.8 mmol), ethyl 3-oxohexanoate (4.71 g, 29.8 mmol) and CsF (452 mg, 2.98 mmol) in Toluene (30 mL) was heated up to 110° C. The orange reaction mixture was stirred over night at 110° C. The flask was then equipped with a small distillation head and short Vigreux column and the reaction mixture was heated while distilling a mixture of EtOH and toluene for 2 h. During the distillation, additional Toluene (15 mL) was introduced into the mixture to prevent it from drying. The reaction mixture was then poured into water (50 mL) and was extracted with MTBE (2×50 mL), washed with water (50 mL) and brine (50 mL), dried over MgSO₄, filtered and concentrated. The crude product was purified by column chromatography on silica gel eluting with a gradient of MTBE in heptane to give (E)-3-methylhex-3-en-2-yl 3-oxohexanoate (3.03 g, 45% yield) as a pale yellow liquid.

¹H NMR (400 MHz, CDCl3, 298 K) b (ppm)=5.47 (tt, J=1.1, 7.1 Hz, 1H), 5.33 (q, J=6.4 Hz, 1H), 3.42 (s, 2H), 2.53 (t, J=7.2 Hz, 2H), 2.04 (quin, J=7.5 Hz, 2H), 1.69-1.58 (m, 6H), 1.33 (d, J=6.6 Hz, 3H), 0.97 (t, J=7.6 Hz, 3H), 0.94 (t, J=7.3 Hz, 3H).

¹³C NMR (101 MHz, CDCl3, 298 K) b (ppm)=202.9, 166.5, 133.0, 129.9, 76.7, 49.7, 44.8, 20.8, 18.9, 16.9, 13.8, 13.6, 11.7.

MS (El, 70 eV): 226 (1, [M]⁺*), 141 (2), 131 (8), 113 (67), 96 (100), 81 (92), 71 (76), 43 (73).

Odour description (10% solution in DPG on paper blotter, 4 h): warm, musky, fatty.

### EXAMPLE 5: (E)-3,5-DIMETHYLHEX-3-EN-2-YL 4-METHYL-3-OXOHEXANOATE

Example 5a: 2,2-dimethyl-5-(2-methylbutanoyl)-1,3-dioxane-4,6-dione: A solution of 2,2-dimethyl-1,3-dioxane-4,6-dione (1.90 g, 13.2 mmol) and pyridine (2.09 g, 2.13 mL, 26.4 mmol) in CH₂Cl₂ (25 mL) was treated with a solution of 2-methylbutanoyl chloride (1.75 g, 14.5 mmol) in CH₂Cl₂ (10 mL) at 5° C. After the addition the cooling bath was removed and the reaction mixture was stirred for 3 h at ambient temperature. The reaction mixture was poured into iced 1M HCl-solution and extracted with CH₂Cl₂ (100 mL), washed with water (100 mL) and brine (100 mL), dried over MgSO₄, filtered and evaporated to give 2,2-dimethyl-5-(2-methylbutanoyl)-1,3-dioxane-4,6-dione (1.35 g, 68% purity, 30% yield) as a yellow liquid which was used in the next step without further purification.

Example 5b: (E)-3,5-dimethylhex-3-en-2-yl 4-methyl-3-oxohexanoate: A solution of 2,2-dimethyl-5-(2-methylbutanoyl)-1,3-dioxane-4,6-dione (1.35 g, 68 wt-%, 4.01 mmol) and (E)-3,5-dimethylhex-3-en-2-ol (1.54 g, 12.0 mmol) in Toluene (13 mL) was heated up to reflux for 4 h. The reaction mixture was then diluted with water (100 mL) and extracted in Ethyl acetate (EtOAc) (2×300 mL), washed with brine, dried over anhydrous sodium sulphate (Na₂SO₄) and concentrated under reduced pressure to obtain the crude. The crude product was purified by column chromatography on silica gel eluting with a gradient of ethyl acetate in heptane to give (E)-3,5-dimethylhex-3-en-2-yl 4-methyl-3-oxohexanoate (0.47 g, 85% purity, 39% yield) as a colorless liquid.

¹H NMR (400 MHz, CDCl3, 298 K, mixture of diastereomers) b (ppm)=5.33-5.22 (m, 2H), 3.45 (s, 2H), 2.64-2.43 (m, 2H), 1.79-1.56 (m, 4H), 1.48-1.35 (m, 1H), 1.31 (d, J=6.5 Hz, 3H), 1.10 (dd, J=1.3, 6.8 Hz, 3H), 0.99-0.87 (m, 9H).

¹³C NMR (101 MHz, CDCl3, 298 K, mixture of diastereomers) b (ppm)=206.5, 166.6, 135.5, 131.4, 77.4, 48.1, 48.0, 48.0, 26.8, 25.6, 25.6, 22.7, 22.7, 18.9, 15.4, 11.8, 11.4.

MS (El, 70 eV): 145 (8), 127 (90), 110 (89), 95 (90), 85 (100), 57 (88).

Odour description (10% solution in DPG on paper blotter, 4 h): fruity, warm, musky.

### EXAMPLE 6: 3,5-DIMETHYLHEX-3-EN-2-YL ETHYL MALONATE

A mixture of diethyl malonate (14 g, 85 mmol) and 3,5-dimethylhex-3-en-2-ol (7.3 g, 57 mmol) was heated to 180° C. while distilling off ethanol. After 30 min, the reaction mixture was cooled down to ambient temperature and subjected directly to column chromatography on silica gel eluting with a gradient of ethyl acetate in heptane to give 3,5-dimethylhex-3-en-2-yl ethyl malonate (5.79 g, 42% yield) as a colorless liquid.

¹H NMR (400 MHz, CDCl3, 300 K, mixture of E and Z isomers) b (ppm)=5.88-4.78 (m, 1H), 4.23-4.13 (m, 2H), 3.37-3.29 (m, 2H), 2.73-2.41 (m, 1H), 1.68-1.56 (m, 3H), 1.33-1.21 (m, 6H), 0.97-0.87 (m, 6H).

¹³C NMR (101 MHz, CDCl3, 296 K, mixture of E and Z isomers) b (ppm)=167.0, 167.0, 166.2, 166.1, 136.9, 135.8, 131.7, 131.1, 77.2, 70.8, 61.8, 61.8, 42.4, 42.3, 27.1, 27.1, 23.7, 23.5, 23.0, 19.3, 19.2, 17.8, 14.4, 14.4, 12.1.

Odour description (10% solution in DPG on paper blotter, 4 h): weak, fruity, green, musky.

### EXAMPLE 7: GREEN, NATURAL PEACH/NECTARINE FRAGRANCE COMPOSITION FOR, E.G, BODY DEODORANT

| | parts by |
|---|---|
| Ingredient | weight |
| Benzaldehyde | 0.1 |
| CASSYRANE ™ (2-tert-butyl-5-methyl-2-propyl-2,5- | 0.1 |
| dihydrofuran) | |
| Beta damascone (1-(2,6,6-trimethyl-1-cyclohexen-1-yl)- | 1 |
| 2-buten-1-one (e + z)) | |
| Gamma Decalactone (decan-4-olide) | 20 |
| Hexenol-3-cis (3-hexen-1-ol (z)-) | 2 |
| Linalool (3,7-dimethyl-1,6-octadien-3-ol) | 20 |
| Manzanate ™ (ethyl 2-methylpentanoate) | 1 |
| Triethyl citrate (TEC) | 55.8 |
| | 100.0 |

By the replacement of 1 part TEC with 1part 3,5-dimethylhex-3-en-2-yl 3-oxohexanoate, the accord fragrance accord above is rounded off (softer and muskier), better blended, and the green note is even more natural, like the crunchy green peach peel.

1. A compound of formula (I) 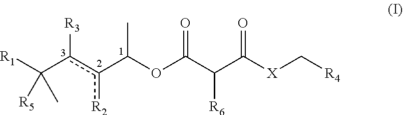
wherein
R₁ and R₃ are independently selected from hydrogen, methyl and ethyl;
R₂ is hydrogen, methyl, ethyl, or methylene;
R₄ is H, or C₁-C₄ alkyl;
R₅ is H, or methyl;
R₆ is selected from hydrogen and methyl;
X is selected from the group consisting of the bivalent residues >CH2, >O, >CHMe, >CMe₂; and
the bond between C-2 and R₂ is a single bond, and the bond between C-2 and C-3 together with the dotted line represents a double bond; or
the bond between C-2 and C-3 is a single bond, and the bond between C-2 and R₂ together with the dotted line represents a double bond; or
the bond between C-2 and R₂ is a single bond, and the bond between C-2 and C-3 is a single bond.

2. The compound according to claim 1 wherein the compound of formula (I) is selected from a compound of formula (Ia) 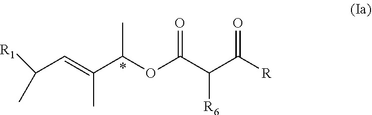
wherein
R₁ is selected from hydrogen, methyl and ethyl;
R₆ is selected from hydrogen, methyl and ethyl; and
R is a hydrocarbon residue comprising 1-6 carbon atoms, optionally comprising one oxygen atom.

3. The compound according to claim 2 wherein the compound is selected from 3,5-dimethylhex-3-en-2-yl 3-oxohexanoate; 3,5-dimethylhex-3-en-2-yl 2-methyl-3-oxohexanoate; (E)-3-methylhex-3-en-2-yl 3-oxohexanoate; (E)-3,5-dimethylhex-3-en-2-yl 4-methyl-3-oxohexanoate; and 3,5-dimethylhex-3-en-2-yl ethyl malonate.

4. A fragranced article comprising as odorant a compound of formula (I) as defined in claim 1, and a consumer product base.

5. A fragranced article according to claim 4 wherein the article is a consumer product base selected from fine fragrance, household products, laundry products, body care products, cosmetic products and air care products.

6. A method of utilizing a compound of formula (I) as defined in claim 1 as a fragrance comprising adding said compound to a consumer product base.

7. A method of improving, enhancing or modifying a consumer product base by adding thereto an olfactory acceptable amount of a compound of formula (I) as defined in claim 1.

8. A fragranced article comprising as odorant a compound of formula (Ia) as defined in claim 2, and a consumer product base.

9. A fragranced article according to claim 8, wherein the article is a consumer product base selected from fine fragrance, household products, laundry products, body care products, cosmetic products and air care products.

10. A method of utilizing a compound of formula (Ia) as defined in claim 2 as a fragrance comprising adding said compound to a consumer product base.

11. A method of improving, enhancing or modifying a consumer product base by adding thereto an olfactory acceptable amount of a compound of formula (Ia) as defined in claim 2.
